(19) United States
(12) Patent Application Publication
(10) Pub. No.: US 2004/0147016 A1
(43) Pub. Date: Jul. 29, 2004

(54) Programmable scaffold and methods for making and using the same

(76) Inventors: Jonathan A. Rowley, Chapel Hill, NC (US); Mohammad A. Heidaran, Cary, NC (US)

Correspondence Address: PATTON BOGGS LLP, 8484 WESTPARK DRIVE, SUITE 900, MCLEAN, VA, 22102, US

(21) Appl. No.: 10/673,438

(22) Filed: Sep. 30, 2003

Related U.S. Application Data
(63) Continuation-in-part of application No. 10/259,817, filed on Sep. 30, 2002.

(51) Int. Cl. C12N 5/00; C12N 5/02

(52) U.S. Cl. 435/325; 435/373

(57) ABSTRACT

The present invention relates to a programmable scaffold, which is a three-dimensional scaffold having interconnected pores and biologically active molecules physically entrapped therein. The scaffold is a lyophilized hydrogel of a polymer. The scaffold can be used in an array on a platform and loaded with various combinations of biologically active molecules for high throughput and parallel screening, as well as tissue engineering. The present invention also relates to methods for making and modifying the scaffolds.

### CROSS REFERENCE TO RELATED APPLICATIONS

This application is a continuation-in-part of U.S. patent application Ser. No. 10/259,817, filed Sep. 30, 2002.

### BACKGROUND OF THE INVENTION

1. Field of the Invention

The present invention relates to scaffolds for cell culture and methods for making and using the same. Particularly, the present invention relates to scaffolds that are programmable with extracellular matrix (ECM) molecules and/or bioaffecting molecules for optimization of microenvironments for cell culture and tissue engineering.

2. Background of the Invention

Cell culture, as an important tool for biological research and industrial application, is typically performed by chemically treating the surface of a cell culture device to support cell adhesion and bathing the adherent cells in culture medium containing supplements for cell growth. “Anchorage dependence” provides that the anchorage-dependent cells would only divide in culture when they are attached to a solid surface; the cells would not divide when they are in liquid suspension without any attachment. The site of cell adhesion enables the individual cell to spread out, capture more growth factors and nutrients, organize its cytoskeleton, and provides anchorage for the intracellular actin filament and extracellular matrix molecules. Thus, a surface that provides sufficient cell adhesion is vital to cell culture and growth.

In addition to cell adhesion and nutrients, hormones and protein growth factors are essential to support mammalian cell growth in cell culture. The requisite hormones and growth factors are contained in serum, which is blood-derived fluid that remains after blood has clotted. Serum contains combinations of growth factors for cell growth. Mammalian cells deprived of serum stop growing and become arrested usually between mitosis and S phase, in a quiescent state called G₀. Various growth factors have been identified and isolated from the serum; however, it is still difficult to make a cell culture substitute that will adequately mimic an in vivo environment. Serum is expensive and needs to be replaced every 1-3 days, as the protein growth factors are quickly taken up by the fast growing cells. Thus, efforts have been made toward developing cell culture systems which promote cell adhesion in the absence of serum.

Tissue engineering is a strategy for regenerating natural tissue. Cell culture in the context of tissue engineering further requires a three-dimensional scaffold for cell support. A scaffold having a three-dimensional porous structure is a prerequisite in many tissue culture applications, such as chondrocyte cell culture, because these cells would otherwise lose their cellular morphology and phenotypic expression in a two-dimensional monolayer cell culture. For regenerating natural tissue, the quality of the three-dimensional matrix can greatly affect cell adhesion and growth, and determine the success of tissue regeneration or synthesis. An optimal matrix material would promote cell binding, cell proliferation, expression of cell-specific phenotypes, and the activity of the cells.

Success in tissue engineering and transplantation of cells depends on the maintenance of the viability, differentiated phenotype, and integration with the body to deliver a desired therapeutic benefit. Maintenance and development of progenitor cells to functional tissue of every type requires different cell types, combination of cell types, physical environment, soluble environment, and proper cell signaling and cell interaction. High throughput and high parallel screening is required to find the suitable combination of microenvironment for tissue development.

A number of porous scaffolds for cell culture and tissue engineering have been disclosed in the literature. Shea et al. (Nature Biotechnology, Vol. 17, pages 551-554 (June 1999)) disclose highly porous three-dimensional poly(lactide-co-glycolide) scaffolds which are made by gas foaming and are entrapped with plasmids. Petronis et al. (Journal of Materials Science: Materials in Medicine, 12, pages 523-528 (2001)) disclose a titania ceramic scaffold with topographic structure at a sub-millimeter scale for hepatocyte in vitro culture; the titania ceramic is microporous, biocompatible, and conducive to cell aggregation. The process for preparing the Petronis et al. scaffold requires repeated oxidation, masking, and etching. Kim et al. (Fibers and Polymers 2001, Vol. 2, No. 2, pages 64-70) disclose a three-dimensional, porous, collagen/chitosan sponge made by lyophilization and crosslinking using EDC and NHS to increase biological stability, and to enhance mechanical properties.

However, none of these scaffolds support cell adhesion. When strong cell adhesion is required, especially for those anchorage-dependent mammalian cell cultures, the scaffolds must be modified to support cell adhesion. To solve the problem, cell adhesion-promoting molecules are immobilized onto the scaffold by covalent binding so that the cells can attach to the ligands. For example, Kobayashi et al. (Biomaterials 1991, Vol. 12 October, 747-751) disclose covalent immobilization of cell-adhesive proteins onto the surface of poly(vinyl alcohol) (PVA) hydrogel by diisocyanates, polyisocyanates, and cyanogen bromide to promote cell adhesion. Kobayashi et al. (Current Eye Research Vol. 10, No. 10, 1991, 899-908) disclose covalent immobilization of cell adhesive proteins and molecules on PVA hydrogel sheets to promote corneal cell adhesion and proliferation. However, covalent modification adds complexity and processing steps that may alter the desirable physical and chemical properties of the scaffold material and the ligands. It has been demonstrated that ECM molecules can randomly adsorb onto hydrophobic polymers such as PGA, PLA, PCL, and all copolymers of polyesters, polyurethane, polystyrene. But, physical adsorption is difficult to control, which makes their use problematic, in processes and assays requiring constant cell adhesion onto a surface.

### SUMMARY OF THE INVENTION

The present invention provides a method for making programmable scaffolds for cell culture, with combinations of molecules promoting cell attachment or having cell signaling functions. The method involves creating a porous scaffold comprising hydrogel, and impregnating this porous scaffold with a solution containing biologically active molecules. Next, the impregnated scaffold is lyphilized or dried so that the biologically active molecules are entrapped within the porous scaffold. The impregnated scaffold is washed to remove salts and pH adjusted, where necessary, prior to lyophilization.

The resultant porous scaffold permits three-dimensional cell or tissue culture and has an interconnected highly porous structure. The porous scaffold can be made from a variety of materials including polymers, ceramics, metal, or composites. These materials can be biocompatible, biodegradable or non-biodegradable. This attribute will depend on the ultimate use for the scaffold.

Acceptable polymers include alginate, hyaluronic acid, agarose, collagen, chitosan, chitin, polytrimethylene carbonate, poly hydroxybutyrate, amino acid-based polycarbonates, poly vinylchloride, polyvinyl alcohol, poly methylmethacrylate, poly fumarate, polyHEMA, polystyrene, PTFE, poly ethylene glycol, or polypropylene glycol-based polymers and derivatives thereof. Biodegradable polymers include poly lactides, glycolides, caprolactones, orthoesters, and copolymers thereof.

The porous scaffold is typically a lyophilized hydrogel of the polymer including, but not limited to, crosslinked alginate, modified alginate, hyaluronic acid or modified hyaluronic acid.

The biologically active molecules include extracellular matrix (ECM) molecules, functional peptides, proteoglycans and glycoproteins capable of signaling cells, growth factors, molecules for optimal cell function, and combinations or derivatives thereof. ECM molecules include fibronectin, laminin, collagen, thrombospondin 1, vitronectin, elastin, tenascin, aggrecan, agrin, bone sialoprotein, cartilage matrix protein, fibronogen, fibrin, fibulin, mucins, entactin, osteopontin, plasminogen, restrictin, serglycin, SPARC/osteonectin, versican, merosin, osteopontin, osteonectin, von Willebrand Factor, heparin sulfate proteoglycan, hyaluronic acid, cell adhesion molecules including cadherins, connexins, selectins, or combination thereof. Growth factors include, but are not limited to, epidermal growth factor, fibroblast growth factor, platelet-derived growth factor, nerve growth factor, transforming growth factor-β, hematopoietic growth factors, interleukins, and combinations thereof. Other growth factors are well known in the art. A combination two or more molecules of ECM and/or growth factor(s) may also be used, which would allow attachment of a specific cell type in close proximity to the growth factor, which would permit the study of the interaction of growth factors and ECM, or permit controlled growth or selection. Accordingly, a microenvironment can be created. More complex microenvironments, comprising several or dozens or even hundreds of different types of biologically active molecules, can also be created The programmable scaffold permits the study of events associated with the triggering of highly specific biological responses in cells through activation or inhibition of signal transduction pathways.

It is also possible with the programmable scaffolds to control and maintain the viability, phenotype, and genetic expression of various cells for a variety of purposes, including tissue engineering, and to use the programmable scaffolds in screening processes including high throughput and parallel screening methods.

The present invention further provides a method for making an array of scaffolds comprising distributing a solution of a suitable polymer on a platform to form solution spots, crosslinking the solution spots to form spots of crosslinked hydrogel, and lyophilizing the spots of crosslinked hydrogel to form an array of scaffolds. The crosslinking reaction mixture may comprise a diamine and a carbodiimide. The carbodiimide can be EDC at an amount of about 25% to about 200% molar ratio of functional groups to hyaluronic acid or alginate, and more particularly, from about 50% to about 100% molar ratio of functional groups to hyaluronic acid or alginate. The diamine, such as lysine or adipic dihydrazide, can be at an amount of about 2% to about 100% molar ratio of functional groups to hyaluronic acid or alginate, and more particularly, about 10% to about 40% molar ratio of functional groups to hyaluronic acid or alginate. The hydrogel solution may further comprise a coreactant including, but not limited to, HoBt, NHS, or sulfo NHS, at a ratio of about 1:50 to 50:1 to the carbodiimide, and preferably, about 1:10 to 4:1 to the carbodiimide (EDC).

The programmable scaffolds and arrays containing the same can be a component of a kit. The kit typically is designed to facilitate use and handling in the context of a desired operation, e.g., cell or tissue culture screening operations. In one embodiment, the kits of the current invention comprise one or more biologically active molecules. In one particular embodiment, the kits of the current invention comprise several biologically active molecules such that a cell culture environment can be customized to the user's specific needs.

### BRIEF DESCRIPTION OF THE DRAWINGS

FIG. 1 depicts the interconnected pore structures of lyophilized hydrogel scaffold of the present invention under a scanning electron microscope.

FIG. 2 shows MTT-stained MC3T3 cells evenly distributed and grown throughout the scaffold of the present invention upon seeding.

FIG. 3 shows cell adhesion and cell growth in the fibronectin-modified scaffold of the present invention, while negative controls, i.e., the unmodified scaffold and the albumin-modified scaffold, do not support cell adhesion or cell growth.

FIG. 4 shows cell adhesion and cell growth in the ECM modified scaffolds of the present invention, while a negative control, i.e., the unmodified scaffold does not support cell adhesion and cell growth.

### DETAILED DESCRIPTION OF THE INVENTION

The present invention provides methods for making scaffolds for cell culture having interconnected pores, and being non-covalently modified with at least one biologically active molecule. These interconnected pore structures guide and support cell and tissue growth. The pore structures provide physical surfaces, onto which the cells can lay their own ECM three-dimensionally. Moreover, the porous structures offer improved nutrient transport to the center of the scaffold and limit the cell cluster size to prevent the formation of large cell clusters that can potentially develop into necrotic centers due to lack of nutrition.

Preferably, the three-dimensional scaffold used in connection with the present invention has a pore size of about 50 to about 700 μm in diameter, in particular, from about 75 to about 300 μm in diameter. The percentage of porosity in the scaffold suitable for the non-covalent modification with the biologically active molecules is about 50% to about 98%, and particularly, about 80% to about 95%.

The scaffold is non-covalently modified with biologically active molecules to provide interactions required for cell growth, or other cellular functions. Within the scaffold, the biologically active molecules are entrapped within the porous structures, but not covalently attached to the polymeric scaffold. The biologically active molecules include, but are not limited to, ECM molecules, functional peptides, proteoglycans and glycoproteins capable of signaling cells, growth factors, and other molecules for optimal cell function, and combination thereof.

When the scaffold of the present invention is functionalized with ECM molecules, it provides support and guidance for cell morphology and tissue development. The native ECM is a non-covalent three-dimensional network of proteins and polysaccharides bound together with cells intermixed. The native ECM is highly hydrated, allows for diffusion, and binds to molecules such as growth factors and cell adhesion molecules to allow for presentation to cells. The present invention provides a biomimetic three-dimensional environment by adding the ECM molecules onto highly hydratable structures, i.e., the lyophilized polysacchride hydrogels.

Entrapped biologically active molecules should be non-toxic, biocompatible, and the scaffold must be highly porous with large and interconnected pores that are mechanically stable to resist cell contraction during tissue development. When the scaffold is non-covalently modified with growth factors, it provides cell interactive signaling for cell growth and cell culture.

The scaffold is made from lyophilization of a hydrogel of a suitable polymer. The polymer is biocompatible, either biodegradable or non-biodegradable. In one embodiment, the scaffold is a lyophilized hydrogel of crosslinked alginate or hyaluronic acid, which is amenable to cell seeding. The pore size and distribution of the scaffold can be adjusted by changing the pH, the concentration of the hydrogel, or the amount of crosslinker.

Alginates are linear, unbranched polymers containing β-(1→4)-linked D-mannuronic acid (M) and α-(1→4)-linked L-guluronic acid (G) residues. Alginates are produced by brown seaweed. Alginates are thermally stable, cold-setting gelling agents that gel in the presence of calcium ions. Such gels can be heat treated without melting, although they may eventually degrade. The alginate polysaccharide hydrogels used in the scaffold of the present invention have several favorable properties: they are easily crosslinked and processed into three-dimensional scaffolds; they have convenient functional groups on the polymer backbone for covalent modification; and the material is non-adhesive to cells in its native state.

Hyaluronic acid is a natural mucopolysaccharide present at varying concentrations in practically all tissues. Aqueous solutions of hyaluronic acid, and the salts or derivatives thereof, or of polysaccharides in general, are characterized by notable viscosity, slipperiness, and the ability to reduce friction.

These polysaccharides can be covalently crosslinked with diamines or dihydrazides as crosslinking molecules and, using the standard carbodiimide chemistry, to initiate the crosslinking reaction when forming the hydrogel. See for example, G. Prestwich et al., Controlled Chemical modification of hyaluronic acid: synthesis, applications, and biodegradation of hydrazide derivatives, J. Controlled Release, 1998, 53, pages 93-103. The hydrogels can be thoroughly washed to remove all reactants, and frozen therein and lyophilized to form the three-dimensional interconnected pore network.

In one embodiment, the scaffolds can be either loosely supplied on the surface of a platform or attached to the surface by covalent attachment. The hydrogel-based scaffold can be covalently attached to the support substrate either via a non-fouling polysaccharide coating at the platform surface, or via amino groups terminating from the substrate surface.

The scaffolds of the present invention are further modified by being impregnated with a solution containing at least one biologically active molecule so that the polymeric hydrogel swells and the biologically active molecule becomes entangled. When the scaffold is impregnated with the solution of biologically active molecule and subsequently lyophilized, the biologically active molecules and the polymer scaffold both collapse to create a interconnected and interpenetrating polymer network that is complex enough to resist re-dissolving of the biologically active molecules. The biologically active molecules thus become physically intertwined within the scaffold. This entanglement may be the basis for controlled release of growth factors and small molecules entrapped therein, while the high molecular weight ECM molecules have polymer chains that are long enough to stably integrate with the hydrogel scaffold and sustain cell adhesion and spreading. Without being bound by theory, the length of the biologically active molecule may be critical for determining its form on the scaffold. If the cell-adhesive molecules are not long enough to physically entangle with the hydrogel network, these molecules may be able to act as anchors for cell adhesion. However, these shorter molecules may be available to act as soluble, control-release factors from the scaffold.

In one embodiment of the current invention, the biologically active molecules comprise fusions proteins. As discussed above, the biologically active molecule that is to be implanted into the scaffold may be too small to become entangled in the scaffold. To accommodate such a situation, if the biologically active molecule is a protein, the protein can be fused to a peptide sequence to produce a peptide that is physically longer and more likely to become entangled into the porous scaffold. The peptide sequence to be fused to the biologically active molecule may itself be a biologically active molecule, resulting in a fusion protein comprising at least two biologically active molecules that are incorporated into the hydrogel scaffold. Alternatively, the peptide sequence to be fused to the biologically active molecule may not be biologically active (i.e., biologically inert) for the particular desired cell culture environment.

Methods of producing fusion proteins are well known in the art typically involves recombinant DNA technology and expression of recombinant DNA in a host cell. As representative examples of appropriate hosts, there may be mentioned: bacterial cells, such as E. coli, Streptomyces, and Salmonella typhimurium; fungal cells, such as yeast; insect cells such as Drosophila S2 and Spodoptera Sf9; animal cells such as CHO, COS or Bowes melanoma; adenoviruses; plant cells, etc. The selection of an appropriate host is deemed to be within the scope of those skilled in the art from the teachings herein.

The recombinant constructs used to make the fusion proteins comprise a vector, such as a plasmid or viral vector, into which a nucleic acid sequence can be inserted, in a forward or reverse orientation. In a preferred aspect of this embodiment, the construct further comprises regulatory sequences, including, for example, a promoter, operably linked to the sequence. Transcription of the DNA encoding the polypeptides of the present invention by higher eukaryotes is increased by inserting an enhancer sequence into the vector. Large numbers of suitable vectors and promoters are known to those of skill in the art and are commercially available.

Promoter regions can be selected from any desired gene using CAT (chloramphenicol transferase) vectors or other vectors with selectable markers. Two appropriate vectors are pKK232-8 and pCM7. Particular named bacterial promoters include lacd, lacZ, T3, T7, gpt, lambda P_{R}, P_{L} and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

The constructs in host cells can be used in a conventional manner to produce the gene product encoded by the recombinant sequence. Alternatively, the polypeptides of the invention can be synthetically produced by conventional peptide synthesizers.

Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., Molecular Cloning: A Laboratory Manual, Second Edition, Cold Spring Harbor, N.Y. (1989), the disclosure of which is hereby incorporated by reference.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), alpha-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Once the scaffold has been hydrated with a solution comprising the biologically active molecules, the scaffold may be washed thoroughly with water or a suitable buffer to adjust pH and remove salts, and then frozen and lyophilized again. The modification does not require covalent bonding. The process is simple, but still adds similar, if not better, biologically active properties to the scaffold. The biologically active molecules can convey information to the cells cultured on the scaffold, and are responsible for cell adhesion interactions on the cultured cells.

The biologically active molecules suitable for entrapment in the scaffold generally have a large molecular weight and a suitable spatial configuration, such that they are intertwined within the scaffold simply entrapped within the porous structures of the scaffold. The biologically active molecules may also be soluble, in which case they can be reversibly entrapped in the scaffold, together with the larger macromolecules. When contacts or interactions occur between the entrapped biomolecules and the cells cultured on the scaffold, such interaction may not be sufficient to pull the entrapped biologically active molecules out of the scaffold.

In one embodiment, the scaffolds can be used to create an array. The arrayed scaffolds can be localized or spread in a continuous manner on the surface of a platform. The platform can be a polystyrene slide or a multiwell plate. The scaffolds can be loosely placed on the platform, such as in the wells of the multiwell plate, or immobilized to the platform, via a derivatized surface, or via a surface coating on the platform. The scaffolds can also be covalently attached to the surface coating. The coating can generally be a non-fouling polysaccharide. The surface may also have amino groups located on the surface that can be covalently linked with the functional groups of the scaffold polymer which has not been used up for crosslinking during the making of the scaffold.

The slide-based scaffold array is particularly useful for testing soluble environments on different non-soluble conditions, such as testing one culture medium condition on combinations of several cell types, or testing different ECMs or peptide components within the scaffolds. The multiwell plate-based microarray is suitable for testing several different drugs on the same engineered tissue-expressing molecules of interest to the pharmaceutical industry, e.g., G-protein coupled receptors, cAMP, cytochrome P450 activity. Furthermore, the arrays of the present invention may be useful, for in vitro screening of several test compounds simultaneously, or testing a single compound against a variety of call types simultaneously. These scaffolds and engineered tissue arrays may be combined and coupled with other apparatus for testing, screening and culture purposes. For example, the array of scaffolds allows for any and all combinations of biologically active macromolecules to be non-covalently added to the scaffolds for both screening of the environments to initiate the specific signaling pathways that direct a desired biological response, such as proliferation, differentiation, angiogenesis, and to mass-produce scaffolds of any one condition for in vivo or in vitro tissue engineering.

The present invention also provides kits. In one embodiment, the kits of the current invention comprise a polymer and at least one biologically active molecule. The polymer would then be crosslinked/hydrated and lyophilized to create the porous scaffold. The biologically active molecules of the kit that are to be incorporated into the hydrogel may be packaged individually and they may be in solution or they may be packaged in a lyophilized form. The solution of the at least one biologically active molecule would then be used to hydrate the lyophilized hydrogel and also to incorporate the biologically active molecule into the scaffold to create a customized cell culture environment. In another embodiment, the kit comprises a pre-formed porous hydrogel scaffold and at least one biologically active molecule, such that the kit user would not need to prepare the hydrogel scaffold prior to incorporating the biologically active molecule(s). The kits may also comprise several, up to dozens, of biologically active molecules such that the programmable scaffolds could be tailored to a larger number of users, based on a wide variety of cell culture environmental needs.

The present invention also provides various methods for assaying the in vivo response or function of cells in response to at least one test molecule. In one embodiment, the test molecule is identical to the biologically active molecule used to prepare the programmable scaffold. The methods of assaying the in vivo response comprise producing a cell culture environment as described herein, with at least one biologically active molecule that is also the test molecule. Cells are then seeded onto the environment and the seeded cell culture environment is then implanted into an in vivo setting. Cell function, proliferation or survival can then be assayed directly or indirectly in response to the test molecule. The cell culture environment may be biopsied prior to the cellular assay. In another embodiment, the biologically active molecule and the test compound are not identical. Test molecules include, but are not limited to, peptide or fragments thereof, polynucleotides, carbohydrates, proteoglycans, glycoproteins, lipids, natural and synthetic polymers, and chemical compounds such as a toxin, a drug and drug candidate.

The current invention also provides methods of removing cells from an in vivo setting into the cell culture environment of the present invention. In one embodiment, prior to implantation, the cell culture environment comprises a biologically active molecule having, or suspected of having, the ability to attract particular cells. The cell culture environment may also comprise a biologically active molecule that can bind the attracted cells once they have entered the scaffold from the subject's body. Thus, this “homing environment” would be implanted, cell-free, into an in vivo setting. After allowing adequate time to for the desired cells to infiltrate the homing environment, the environment would then be removed from the subject and the newly infiltrated cells could then be isolated in an in vitro setting and subsequently cultured. Alternatively, once the homing environment has been seeded with cells in vivo, the environment could be removed from the patient and re-implanted into another location of the same patient or into a different patient altogether. In one particular embodiment of the current invention, the biologically active molecule of the homing environment could be used to attract stem cells of various types, such as, but not limited to, liver stem cells, hematopoeitic stem cells, neuronal stem cells, cardiac stem cells, islet stem cells, mammary stem cells and bone marrow stem cells.

The methods for making the scaffold and microarray of the present invention are described in further details in the examples. The following examples are illustrative, but do not limit the scope of the present invention. Reasonable variations, such as those occur to reasonable artisan, can be made herein without departing from the scope of the present invention.

### EXAMPLES

### Example 1

### Making the Porous Scaffold of the Present Invention

Three grams of alginate (MVG alginate, ProNova, Norway) were slowly dissolved in 100 ml MES buffer (pH 6.0) to obtain 3% w/v alginate solution (or pH 6.5 for use of lysine).

Sulfo-N-hydroxysuccinimide (Sulfo-NHS) 164 mg (MW217.13, Sigma) and 100 mg Adipic Acid Dihydrate (AAD, MW 174) were added into 50 ml 3% w/v alginate solution to obtain 15% crosslinking.

The alginate solution (25 ml) was poured into a 50 ml conical flask, and 365 mg of 1-ethyl-3-(3-dimethyl aminopropyl) carbodiimide (EDC, MW 191.7, Pierce) was quickly added to initiate crosslinking reaction.

The solution was quickly poured into an inverted petri dish with the top upside down and 2 mm spacers at sides with an inverted bottom. This provided parallel surfaces separated by the 2 mm gap to gel alginate with homogeneous thickness. The material was allowed to gel overnight.

The hydrogel formed and was punched into several 6 mm×2 mm disks by a 6 mm biopsy punch. The gel disks were rinsed in deionized water for 3 hours with 5 water changes to leach salts and reactants. The gel disks were then placed on plastic surface and frozen at −70° C. for 4 hours, and lyophilized overnight to obtain three-dimensional porous scaffolds of the present invention.

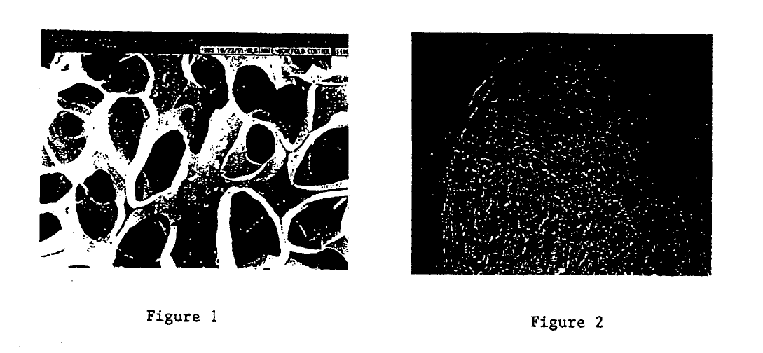

As indicated in FIG. 1, the three-dimensional scaffold was obtained with interconnected pore structures, which was useful for further modification with bioaffecting molecules in the present invention. It was possible that the porous structures were originated from ice crystals formed during freezing, and when the ice crystals were lyophilized, the space left by the ice crystals formed interconnected porous structures. The carboxy (—COOH) groups in the hydrogel that were not crosslinked during the reaction might provide potential sites for further modification of the scaffolds.

### Example 2

### Making the Porous Scaffold of the Present Invention

Two percent (w/v) alginate solution and 2% (w/v) hyaluronic acid (HA) solution in 0.1 M MES buffer (pH 6.0) were added with solution of HoBt and AAD, respectively, at 110 mg AAD/50 ml alginate/HA solution. Next, EDC dissolved in 0.1 MES buffer was added to alginate solution or hyaluronic acid solution to initiate crosslinking reactions, respectively, at 195 mg EDC/10 ml alginate/HA. The solution was then quickly poured into a container and allowed to gel overnight.

Hydrogels formed in the container and were punched into several 6 mm×2 mm disks. The gel disks were rinsed in water and PBS buffer to leach out salts and reactants. The gel disks were frozen and lyophilized overnight.

The three-dimensional scaffold was obtained with interconnected pore structures as lyophilized hydrogels of crosslinked alginates or hyaluronic acids. The carboxy (—COOH) groups in the hydrogel that were not crosslinked during the reaction might provide potential sites for further modification of the scaffolds. The scaffolds with interconnected pores were useful for further modification with bioaffecting molecules in the present invention.

### Example 3

### Making the Microarray of the Present Invention

After following Steps 1-3 of Example 1, the gelling solution was dispensed into wells of a 50-well silicone gasket fitted onto HA-coated polystyrene slide. Alginate hydrogel not only crosslinked in a three-dimensional arrayed configuration but also crosslinked with the surface of the slide. If the alginate gelled before all 50 wells could be filled with the gelling solution, one might slow down the gelling process by increasing pH or adding reactants at different times. The slide was frozen and lyophilized.

The three-dimensional scaffolds were arrayed and covalently attached to the slide surface which allowed for high parallel and high throughput screening and cell culture.

### Example 4

### Making the Microarray of the Present Invention

Alginate (MVG alginate, ProNova, Norway) solution 2% (w/v) was obtained by slowly dissolving alginates in 0.1 M MES buffer (pH 6.5). Hydroxyl benzotiazole 68.3 mg (HoBt, H-2006, Sigma) and 110 mg AAD were added into 50 ml 2% w/v alginate solution to obtain 25% crosslinking of the carboxy groups. The alginate solution aliquot in 3 ml volume was poured into a 10 ml plastic tube for reaction. The top of the tube was cut off so that the pipette tip could fit to bottom. EDC 58 mg (MW 191.7, Pierce) was added into 3 ml 2% alginate solution to initiate the crosslinking reaction. The alginate solution was quickly aspirated and dispensed into wells of the 50-well gaskets placed onto 0.5% or 1.0% alginate-coated slides, repeating the dispense 2-3 times in the same well without going over the lip of the well. The pH of solution was adjusted for varying crosslinking reaction rate.

The slides loaded with gelling alginate solution were allowed to gel for about 20-60 minutes. Gaskets could be stacked for thicker gels. The slides were frozen at −70° C. for several hours or overnight and lyophilized until dry.

Scaffolds arrayed completely on the slide. Increased pH slowed down the gelling kinetics enough to allow handling of the solution prior to gelling. The gaskets were removed in most cases without disrupting the gels and keeping the gels stuck to the surface of the slide. Completely arrayed three-dimensional scaffolds of the present invention were obtained.

### Example 5

### Making the Microarray of the Present Invention

The steps of Example 4 were repeated; however, the pH of the alginate solution aliquots was adjusted to 5.5, 6.0, 6.5, and 7.0 before EDC was added to initiate the crosslinking reaction. Quality and time of the gelling process were observed and recorded. Specifically, the alginate solution with pH 7.0 obtained a good balance between gelling quality and gelling time.

### Example 6

### Seeding Cells on the Microarray of the Present Invention

MC3T3 cells in suspension at 1×10⁶ cells/ml were seeded onto the scaffolds. A cell suspension of 10 μl was seeded onto the scaffolds, with each scaffold having a diameter of 3 mm and a thickness of 1 mm (volume was about 7 μl). Three scaffold arrays were attached to the bottom of a 100 mm petri dish, and left under the laminar flow hood UV source for 20-30 minutes for sterilization.

The cell suspension entered the scaffolds due to capillary action and the cells were distributed throughout the pores of the scaffolds. Twenty ml of 10% FBS containing medium was added to the petri dish containing the slides for cell culture.

After 48 hours, cells were stained by MTT and digital images were recorded. Cells could also be observed under confocal microscope and phase contrast microscope.

As shown in FIG. 2, cells seeded on the arrayed scaffolds of the present invention were evenly distributed throughout the scaffold and cells easily entered the open pore structures of the lyophilized scaffolds without interaction with the alginate scaffold. The figure also demonstrates the interconnectivity of the scaffolds.

### Example 7

### Seeding Cells on Unmodified Scaffolds

Cell suspensions of MC3T3 cells were prepared at 0.5, 1.0, 5.0, and 1×10⁶ cells/ml. Aliquots of 60 μl of the cell suspensions were seeded onto each scaffold (56.5 μl in volume) of a microarray on a 24-well plate by placing a tip of a the pipet, loaded with cell suspension, in the middle of the scaffold and dispensing the cell suspension into the scaffold.

Culture medium (0.5 ml) was added to each well and cells were cultured under proper conditions. Twenty hours later, cells were stained with MTT 10% (v/v) for observation.

Seeded cells were distributed throughout the alginate scaffold along the entire thickness, and the cells existed mainly as clumps of cells. As the focal plane was changed on the microscope, new cell aggregates appeared in focus. The adhesion of the cells to each other was most likely due to cells not being able to adhere to the alginate scaffold.

Initial cell concentration and porous structure of the scaffolds had effects on cell seeding distribution. The smaller the pore, the more the cell aggregates with fewer cells than the aggregates in the larger-pored scaffolds. The larger pored scaffolds had larger clumps of cells and fewer in number. It demonstrated that the three-dimensional scaffolds of the present invention were useful for cell seeding and three-dimensional cell growth and cell culture.

### Example 8

### Cell Culture on Modified Scaffold of the Present Invention

Scaffolds of lyophilized hydrogels of crosslinked alginate and hyaluronic acid were impregnated with 0.1 mg/ml collagen I solution in acid buffer. The impregnated scaffolds were either unwashed or washed in PBS and water for 4 hours. Washed or unwashed scaffolds were frozen at −70° C. for several hours and lyophilized.

Trypsinized MC3T3 cells (50 μl) at 4×10⁶ cells/ml were seeded onto each scaffold by P200 Pipetteman to obtain a cell density of approximately 200,000 cells per scaffold. The cell suspension was filled in the pipet tip, and when the end of the tip penetrated the scaffold, the cell suspension was simultaneously injected into the scaffold. The scaffolds seeded with cells were transferred into a plate with 200 μl culture medium (aMEM+10% FBS) and maintained at 37° C. in an incubator and observed continuously.

Cells might be trypsinized and collected for count for cell growth. Alternatively, cells grown on the scaffolds were observed under the microscope and sampled every day for examination on cell morphology and cell growth. The scaffolds with cells grown thereon were stained by conventional methods for cell viability such as MTT. Cell suspensions that were not seeded on any scaffold were observed under the same conditions as a control. Kit L-3224 by Molecular Probes was used to assay for cell viability.

Cell attachment and cell growth were observed on the alginate or hyaluronic acid scaffold modified with collagen of the present invention, while the scaffolds absent collagen did not support cell attachment and cell growth. Cells attached and spread on the modified scaffold, which was necessary for cell proliferation; while cells in unmodified scaffolds existed as multicellular aggregates because they could not adhere to the scaffolds.

Scaffolds with non-covalently modified ECM molecules of the present invention supported cell adhesion and cell growth, while the unmodified scaffolds did not support cell adhesion and cell growth. The non-covalent modification method of the present invention thus promoted cell function, such as cell attachment and cell growth.

### Example 9

### Cell Culture on the Modified Scaffold of the Present Invention

Hydrogel alginate scaffolds were modified with fibronectin (Human fibronectin in PBS, from Becton Dickinson Labware) or Bovine serum albumen (BSA, fraction V, Sigma IIA-7906). Fibronectin is an ECM protein known to promote cell adhesion and cell attachment, while BSA, a large protein similar to fibronectin in size, does not have any known properties that promote cell adhesion and cell attachment. The concentrations of fibronectin or BSA solutions for impregnating the scaffolds were both 100 μg/ml. After being impregnated with the solutions, the scaffolds were frozen and lyophilized.

The scaffolds were then seeded with MC3T3 cells at 100,000 cells/scaffold. The scaffolds seeded with cells were cultured under proper conditions and observed continuously and stained by MTT at the end for cell viability.

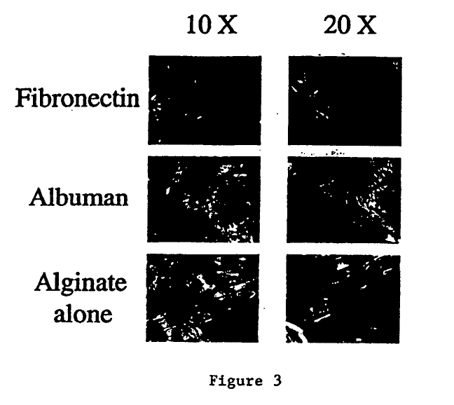

As shown in FIG. 3, cell attachment was observed on the fibronectin-modified scaffolds of the present invention, while the scaffolds albumin-modified scaffolds and the unmodified scaffolds did not support such cell attachment or promote cell adhesion. Cell growth was also observed on the fibronectin-modified scaffolds of the present invention, while the albumin-modified scaffolds and the unmodified scaffolds did not support cell growth.

### Example 10

### Cell Culture in the Arrayed Scaffolds of the Present Invention

Polystyrene slides were coated with polyethyleneimine (PEI) and hyaluronic acid (HA). Masks from Grace Biolab were used to array EDC/AAD crosslinked HA scaffolds.

The lyophilized scaffold arrays were hydrated with solutions containing ECM molecules including human fibronectin (100 μg/ml, BD Labware), mouse laminin (100 μg/ml, BD Labware) and Collagen IV (100 μg/ml, BD Labware), respectively. Then, the hydrated scaffold arrays were frozen and lyophilized to obtain the modified scaffold arrays.

Next, the MC3T3 cells were seeded (2×10⁶ cells/ml), onto the scaffold. The slide reservoir was filled with 5 ml of culture medium and cultured for 3-4 days. Cells were stained with MTT for viability. The cells were also stained with propidium iodide for fluorescent staining of the nuclei, and observed under the Universal Imaging System for photograph.

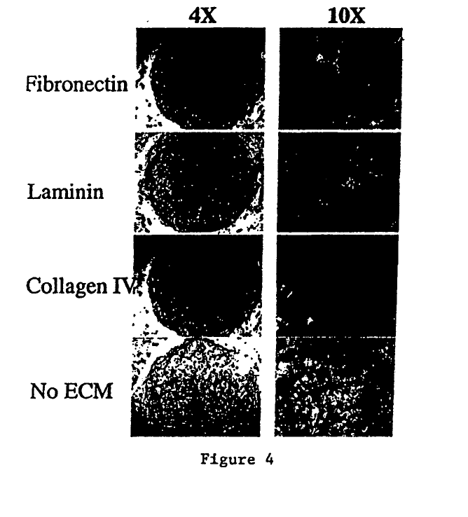

As shown in FIG. 4, cells attached to the modified scaffolds of the microarray of the present invention, but no cell attachment or cell growth was observed on the unmodified scaffolds. ECM molecule-modified scaffolds of the present invention supported cell adhesion and cell growth, and these modified scaffolds, when in an array, were useful in screening for microenvironments that promote for cell attachment, cell signaling and/or cell growth.

### Example 11

### Cell Culture in the Modified Arrayed Scaffolds of the Present Invention

Arrayed alginate scaffolds of the present invention were modified with human fibronectin at 100 μg/ml, or mouse laminin (Gibep) at 100 μg/ml, or Matrigel (Becton Dickinson) at 50 μg/ml. ECM or Matrigel solution (1 μl) was used to impregnate each scaffold.

Matrigel™ is a commercially available (Becton Dickinson Bioscience) solution of solubilized basement membrane preparation extracted from Engelbreth-Holm-Swarm (EHS) mouse sarcoma, which is a tumor rich in extracellular matrix components. The major component of Matrigel™ is laminin, followed by collagen IV, entactin, and heparan sulfate proteoglycan. Matrigel™ also contains TGF-β fibroblast growth factor, tissue plasminogen activator, and other growth factors which occur naturally in the EHS tumor. At room temperature, Matrigel™ polymerizes to produce biologically active matrix material resembling the mammalian cellular basement membrane. Thus, Matrigel™ is effective for the attachment and differentiation of both normal and transformed anchorage dependent epithelial and other cell types, including but not limited to, neurons, hepatocytes, Sertoli cells, mammary epithelial, melanoma cells, vascular endothelial cells, thyroid cells and hair follicle cells.

The scaffolds were seeded with HEPG2 cells or MC3T3 cells at 100,000 cells per scaffold and cultured in 10% serum-containing medium for 1 week. The scaffolds were maintained and observed continuously. Cells were stained by MTT for cell viability and also recorded by phase contrast microscopy.

ECM-or Matrigel-modified scaffolds of the present invention supported cell adhesion and cell growth of cells from different tissue (hepatocytes and osteoblasts) and different species (mouse and human). The array of the modified scaffolds allowed parallel and high throughput screening for microenvironments for cell culture for different cell types as well as for different cell culture environments.

### Example 12

### Cell Culture in the Modified Arrayed Scaffolds of the Present Invention

Arrayed alginate scaffolds of the present invention were modified with human fibronectin at 100, 30, 10, 3, and 1 μg/ml in PBS, or with mouse laminin (Gibco) at 100, 30, 10, 3, and 1 μg/ml in PBS, or with mouse collagen IV at 100, 30, 10, 3, and 1 μg/ml.

The scaffolds were seeded with cells at 100,000 cells per scaffold, cultured, and observed continuously. ECM-modified scaffolds of the present invention supported cell adhesion and cell growth of cells at various concentrations. The array of the modified scaffolds allowed parallel and high throughput screening for microenvironments for cell culture for different cell types as well as for different cell culture environments.

What is claimed is:

1. A method of making a cell culture environment, said method comprising:
a) crosslinking a polymer to form a hydrogel;
b) forming pores within said hydrogel; and
c) non-covalently incorporating at least one biologically active molecule into said porous hydrogel.

2. The method of claim 1, wherein said polymer is selected from the group consisting of alginate, modified alginates, hyaluronic acid, modified hyaluronic acid, agarose, collagen, chitosan, chitin, poly vinyl alcohol, polytrimethylene carbonate, poly hydroxybutyrate, amino acid-based polycarbonates, poly vinylchloride, polyHEMA, PTFE, poly ethylene glycol, poly methylmethacrylate, poly fumarate, polypropylene glycol-based polymers, and derivatives thereof.

3. The method of claim 1, wherein said forming pores comprises freezing and lyophilizing said hydrogel.

4. The method of claim 3, wherein said non-covalently incorporating said at least one biologically active molecule comprises hydrating said lyophilized hydrogel with a solution comprising said at least one biologically active molecule, and drying or lyophilizing said hydrated porous hydrogel.

5. The method of claim 1, wherein said at least one biologically active molecule is selected from the group consisting of extracellular matrix molecules (ECM), growth factors, cell-signaling molecules and derivatives thereof.

6. The method of claim 5, wherein said at least one biologically active molecule comprises at least one extracellular matrix molecule (ECM) and at least one cell signaling molecule.

7. The method of claim 6, wherein said ECM is selected from the group consisting of fibronectin, laminins, collagens, thrombospondin 1, vitronectin, elastin, tenascin, aggrecan, agrin, bone sialoprotein, cartilage matrix protein, fibronogen, fibrin, fibulin, mucins, entactin, osteopontin, plasminogen, restrictin, serglycin, SPARC/osteonectin, versican, von Willebrand Factor, heparin sulfate proteoglycan, hyaluronan, merosin, osteopontin, osteonectin, cell adhesion molecules, cadherins, connexins and selectins.

8. The method of claim 5, wherein said growth factor is selected from the group consisting of acidic fibroblast growth factor, basic fibroblast growth factor platelet-derived growth factor, nerve growth factor, transforming growth factor-β, hematopoietic growth factors and interleukins.

9. A cell culture environment, comprising a porous hydrogel scaffold and at least one biologically active molecule, wherein said at least one biologically active molecule is non-covalently attached to said porous hydrogel scaffold.

10. The cell culture environment of claim 9, wherein said hydrogel comprises a polymer selected from the group consisting of alginate, modified alginate, hyaluronic acid, modified hyaluronic acid, agarose, collagen, chitosan, chitin, poly vinyl alcohol, polytrimethylene carbonate, poly hydroxybutyrate, amino acid-based polycarbonates, poly vinylchloride, polyHEMA, PTFE, poly ethylene glycol, poly methylmethacrylate, poly fumarate, polypropylene glycol-based polymers, and derivatives thereof.

11. The cell culture environment of claim 9, wherein said hydrogel is covalently crosslinked.

12. The cell culture environment of claim 9, wherein said at least one biologically active molecule is selected from the group consisting of extracellular matrix molecules (ECM), growth factors, cell-signaling molecules, and derivatives thereof.

13. The cell culture environment of claim 12, wherein said at least one biologically active molecule comprises at least one extracellular matrix (ECM) molecule and at least one growth factor.

14. The cell culture environment of claim 13, wherein said ECM is selected from the group consisting of fibronectin, laminins, collagens, thrombospondin 1, vitronectin, elastin, tenascin, aggrecan, agrin, bone sialoprotein, cartilage matrix protein, fibronogen, fibrin, fibulin, mucins, entactin, osteopontin, plasminogen, restrictin, serglycin, SPARC/osteonectin, versican, von Willebrand Factor, heparin sulfate proteoglycan, hyaluronan, merosin, osteopontin, osteonectin, cell adhesion molecules, cadherins, connexins and selectins.

15. The cell culture environment of claim 13, wherein said growth factor is selected from the group consisting of acidic fibroblast growth factor, basic fibroblast growth factor platelet-derived growth factor, nerve growth factor, transforming growth factor-β, hematopoietic growth factors and interleukins.

16. An array, comprising the cell culture environment of claim 9.

17. The array of claim 16, wherein said array comprises more than one said cell culture environment, each of said more than one cell culture environments being composed of identical constituents.

18. The array of claim 16, wherein said array comprises more than one said cell culture environment, each of said more than one cell culture environments being composed of different constituents.

19. A method of culturing cells, comprising:
a) seeding cells on the cell culture environment of claim 9; and
b) maintaining said cells within said environment under appropriate cell culture conditions.

20. A method for assaying cellular function in response to at least one test molecule, said method comprising:
a) seeding cells onto the cell culture environment of claim 9, wherein said at least one non-covalently attached biologically active molecule is said test molecule;
b) maintaining said cells on said cell culture environment under appropriate conditions; and
c) determining said cultured cells' response to said maintenance on said cell culture environment.

21. The method of claim 20, wherein said cells are seeded in an in vitro setting.

22. The method of claim 21, wherein said appropriate conditions comprise cell culture conditions.

23. The method of claim 20, wherein said cells are seeded in an in vivo setting.

24. The method of claim 23, wherein said maintaining said cells under appropriate conditions comprises maintaining said cell culture environment in a subject.

25. A method of producing a cell-based in vivo transplant, said method comprising:
a) in an in vitro setting, seeding cells on the cell culture environment of claim 9; and
b) maintaining said cells on said cell culture environment under appropriate cell culture conditions.

26. A kit, comprising:
a) a polymer, said polymer having the ability to form a porous hydrogel; and
b) at least one biologically active molecule,
said kit being used to create a cell culture environment.

27. The kit of claim 26, wherein said polymer exists as a porous hydrogel.
